(19) United States
(12) Patent Application Publication
(10) Pub. No.: US 2020/0299670 A1
(43) Pub. Date: Sep. 24, 2020

(54) POLYMERIC BIOCATALYSTS AND METHODS

(71) Applicants: UCHICAGO ARGONNE, LLC, Chicago, IL (US); BOARD OF TRUSTEES OF NORTHERN ILLINOIS UNIVERSITY, DeKalb, IL (US)

(72) Inventors: Tao Li, Downers Grove, IL (US); Gyorgy Babnigg, Countryside, IL (US); Jessica L. Johnson, Plainfield, IL (US); Erik Sarnello, DeKalb, IL (US)

(21) Appl. No.: 16/826,135

(22) Filed: Mar. 20, 2020

Related U.S. Application Data
(60) Provisional application No. 62/821,937, filed on Mar. 21, 2019.

(51) Int. Cl. C12N 11/084; C12N 11/096; C12N 9/42; C12N 9/18

(52) U.S. Cl. CPC C12N 11/084; C12N 11/096; C12N 9/2445; C12Y 302/01004; C12N 9/18; C12Y 302/01021; C12N 9/2437

(57) ABSTRACT

Biocatalysts disclosed herein can have a core and a shell, with the core including a polymer having a pyridine functional group, and the shell including an enzyme that interacts with the polymer. The biocatalysts disclosed herein can have improved stability, control, and activity as compared to the enzyme in a free, non-interacted state.

### CROSS-REFERENCE TO RELATED APPLICATIONS

This application claims the benefit of the filing date of U.S. Provisional Application No. 62/821,937, filed Mar. 21, 2019, the disclosure of which is hereby incorporated by reference in its entirety.

### STATEMENT REGARDING FEDERAL SPONSORED RESEARCH OR DEVELOPMENT

This invention was made with government support under Contract No. DE-AC02-06CH11357 awarded by the United States Department of Energy to UChicago Argonne, LLC, operator of Argonne National Laboratory. The government has certain rights in the invention.

### BACKGROUND

### Field of the Disclosure

The disclosure relates to biocatalysts having a polymeric core containing a pyridine functional group, and a shell comprising an enzyme, and methods of making same.

### Brief Description of Related Technology

Enzymes are efficient biocatalysts, enabling faster reactions with more energy efficiency. Different from conventional chemical catalysts, biocatalysts are nature-approved, biodegradable, and functional under mild conditions in water with high selectivity. Enzymes have been widely used to convert chemicals to useful fuels and drugs, treat disease, nerve agent detoxification, and aid in environmental decontamination. However, enzymes cannot always compete economically with traditional chemistry and inorganic catalysts. Major challenges preventing widespread use of enzymatic biocatalysts include the cost of production, low loading, limited stability (and therefore, limited activity), and separation difficulties. For commercial-scale processes, enzymes are often immobilized on solid supports so that they can be reused. A major challenge is that modified enzyme activity and stability are not comparable to that of the enzyme in a free, non-interacted state, and enzyme recovery can be difficult.

However, enzyme immobilization methods can have various drawbacks. They are not universal for all enzymes, have poor immobilization yield, drastically reduce enzyme activity, and can involve the use of expensive support materials. Typically, there are three different ways for the enzyme immobilization: binding to a support through the physical adsorption, chemical crosslinking, and encapsulation. However, each method is selective for certain enzymes and each has its disadvantage.

Although many examples of enzyme immobilization exist, a general methodology has yet to be developed.

### SUMMARY

There is a need in the art for a general immobilization protocol that can be applied readily and inexpensively across multiple enzyme classes. In embodiments, a method of forming a biocatalyst having a core surrounded by a shell can include admixing an aqueous phase comprising an enzyme with a dispersed phase comprising a polymer having a pyridine functional group under conditions sufficient to disperse droplets of the dispersed phase in the aqueous phase, wherein the enzyme interacts with the polymer and arranges at the interface between the droplets of the dispersed phase and the aqueous phase to form the shell, wherein after forming the shell the enzyme can have an activity that is at least 80% of the activity of the enzyme in a free, non-interacted state. The enzyme can be selected from the group consisting of an oxidoreductase, a transferase, a hydrolase, a lyase, an isomerase, a ligase, and any mixture thereof. The enzyme can be selected from the group consisting of carbohydrate-active enzymes, cellulase, and any mixture or combination thereof. The enzyme can also be selected from lipases.

In embodiments, a biocatalyst can include a core comprising a polymer having a pyridine functional group, and a shell comprising an enzyme, wherein the enzyme covalently and/or non-covalently interacts with the polymer on the surface of the core. The biocatalyst can have (a) a melting point that is at least equal to the melting point of the enzyme in a free, non-interacted state, and (b) an enzymatic activity after 200 days at room temperature that is at least about equal to the enzymatic activity of the enzyme in a free, unreacted state.

In embodiments, a method can allow enzyme conformation and functionality to be preserved by assembling the polymers and enzyme into a hierarchal structure. For various embodiments, in situ X-ray scattering was used to study the co-assembly process of P4VP and protein. It was observed that in embodiments, once protein and polymer were mixed, the assembly occurred immediately, and the protein retained their spherical shape on the surface of the P4VP. In various experiences, several beta-glucosidases were purified on a scale of several hundred mg and their activities and thermal stabilities were tested. A beta-glucosidase was observed to assemble with P4VP to form polymer/protein core/shell structures, which were characterized by DLS, SAXS and TEM. In embodiments, it was observed that solvents can affect the activity of the enzymes. In one experiment, give different water-soluble solvents were tested, including THF, DMS, DMSO, methanol and ethanol. It was found that at methanol concentrations of up to 30% the enzyme retained its activity, but lost activity for the other tested solvents at such concentrations.

Further aspects and advantages of the disclosure will be apparent to those of ordinary skill in the art from a review of the following detailed description. While the compositions and methods are susceptible of embodiments in various forms, the description hereafter includes specific embodiments, with the understanding that the disclosure is illustrative, and is not intended to limit the scope of the disclosure to the specific embodiments described herein.

### BRIEF DESCRIPTION OF THE DRAWINGS

FIG. 1 is a graph showing the activity of a biocatalyst as described herein as compared to the activity of a corresponding enzyme in a free, non-interacted state.

FIG. 2 is an SDS-PAGE image of the stability of an enzyme in a free, non-interacted state after storage for over 200 days at 4° C. and room temperature.

FIG. 3A is a TEM image of a biocatalyst according to embodiments of the disclosure.

FIG. 3B is a TEM image of the biocatalysts according to embodiments of the disclosure.

FIG. 4 is a graph illustrating the effect of various salt concentrations on the activity of a biocatalyst according to embodiments of the disclosure.

FIG. 5 is a graph illustrating the activity of a biocatalyst as described herein in various solvents and solvent concentrations.

FIG. 6 is a graph illustrating the melting point of a biocatalyst as described herein as compared to the melting point of a corresponding enzyme in a free, non-interacted state.

FIG. 7A is a TEM image of a biocatalyst according to embodiments of the disclosure.

FIG. 7B is a TEM image of a biocatalyst according to embodiments of the disclosure wherein the enzymes are bound to 15 nm gold nanoparticles.

FIG. 7C is a TEM image of a biocatalyst according to embodiments of the disclosure wherein the enzymes are bound to 15 nm gold nanoparticles, showing the nanoparticles are bound to the surface of the biocatalyst.

FIG. 7D is a TEM image of a biocatalyst according to embodiments of the disclosure wherein the enzymes are bound to 15 nm gold nanoparticles, showing the nanoparticles are bound to the surface of the biocatalyst.

FIG. 7E is a TEM image of a biocatalyst according to embodiments of the disclosure wherein the enzymes are bound to 15 nm gold nanoparticles, showing the nanoparticles are bound to the surface of the biocatalyst.

FIG. 7F is a TEM image of a biocatalyst according to embodiments of the disclosure wherein the enzymes are bound to 15 nm gold nanoparticles, showing the nanoparticles are bound to the surface of the biocatalyst.

FIG. 7G is a TEM image of a biocatalyst according to embodiments of the disclosure wherein the enzymes are bound to 15 nm gold nanoparticles, showing the nanoparticles are bound to the surface of the biocatalyst.

FIG. 7H is a TEM image of a biocatalyst according to embodiments of the disclosure wherein the enzymes are bound to 15 nm gold nanoparticles, showing the nanoparticles are bound to the surface of the biocatalyst.

FIG. 8A is a graph of the DLS log normal size distribution for the removal of solvent via evaporation.

FIG. 8B is a graph of the DLS log normal size distribution for the removal of solvent via dialysis.

FIG. 9 is a 3D-plot of average biocatalyst radii when varying the concentrations of bovine serum albumin (BSA) and polymer.

FIG. 10 is a graph of the fluorescence spectra of three different assemblies of biocatalysts according to embodiments of the disclosure.

FIG. 11A is TEM image of a biocatalyst according to embodiments of the disclosure.

FIG. 11B is a graph of the DLS log normal size distribution of biocatalysts prepared with an enzyme and various concentrations of polymer.

### DETAILED DESCRIPTION

The disclosure is generally directed to biocatalysts. Disclosed herein are methods that allow enzyme conformation and functionality to be preserved by assembling polymers and enzyme(s) into a hierarchical structure. In embodiments, advantageously, upon mixing enzyme and polymer, assembly of the biocatalysts occurs immediately, and the enzyme maintains its spherical shape on the surface of polymer. In embodiments, the biocatalysts are a reusable biocatalyst, which have equal or enhanced enzymatic activity while having improved recoverability and stability of the enzyme(s). As used herein, “a reusable biocatalyst” means that the biocatalyst can be recharged with enzyme for further use after the enzyme originally assembled on the surface of the polymer is exhausted. For example, after the enzyme assembled on the surface of the polymer is exhausted, the polymer can be mixed with a second enzyme (that is the same or different from the first enzyme) to recharge the biocatalyst, thereby making it reusable.

### Biocatalyst Composition

Disclosed herein are biocatalysts comprising a core and a shell. In embodiments, the core can include a polymer. In embodiments, the polymer can have a pyridine functional group. In embodiments, the shell can include an enzyme. The enzyme can be bound to the surface of the core, for example, via interactions with the pyridine functional group(s) of the polymer.

In embodiments, the polymer can have one or more pyridine functional groups selected from poly(4-vinyl pyridine), poly(2-vinyl pyridine), and any copolymer or combination thereof. In embodiments, the pyridine functional group can be poly(styrene-b-4-vinyl pyridine), poly(styrene-b-2-vinyl pyridine), poly(2-vinyl pyridine-b-ε-caprolactone), poly(ethylene oxide-b-4-vinyl pyridine), poly(styrene-b-4-vinyl pyridine-b-styrene), poly(4-vinyl pyridine-b-styrene-b-4-vinyl pyridine), poly(styrene-b-4-vinyl pyridine-b-ethylene oxide), and any combination thereof. In embodiments, the polymer can include or can be a poly(4-vinyl pyridine). In embodiments, the polymer can include or is a poly(2-vinyl pyridine-b-ε-caprolactone). In embodiments, the polymer can include or is the combination of poly(4-vinyl pyridine) and poly(2-vinyl pyridine-b-ε-caprolactone).

The polymer can be present in a concentration of about 0.1 mg/mL to about 20 mg/mL, for example at least about 0.1, 0.5, 0.7, 1, 2, 4, 5, 6, 8, 10, or 12 mg/mL and/or up to about 20, 18, 17, 15, 13, 10, 8, 6, or 4 mg/mL. In embodiments, the polymer is present in a concentration of about 0.5 mg/mL to about 15 mg/mL, about 1 mg/mL to about 10 mg/mL, or about 2 mg/mL to about 6 mg/mL.

The polymer can have a molecular weight (Mw) ranging from about 20 kDa to about 250 kDa, for example at least about 20, 25, 30, 35, 40, 45, 50, 55, 60, 65, 70, 75, 80, 85, 90, 95, 100, or 110 kDa and/or up to about 250, 225, 200, 175, 150, 140, 130, 120, 110, 100, or 90 kDa. In embodiments, the polymer can have a molecular weight ranging from about 50 kDa to about 100 kDa.

The enzyme is not particularly limited. In embodiments, the enzyme is selected from the group consisting of an oxidoreductase, a transferase, a hydrolase, a lyase, an isomerase, a ligase, and any mixture thereof. In embodiments, the enzyme is one or more carbohydrate-active enzyme, cellulase, lipase, and any mixture or combination thereof. In embodiments, the enzyme is a carbohydrate-active enzyme (CAZyme). For example, in embodiments, the CAZyme is one or more of glycoside hydrolases, glycosidetransferases, polysaccharide lyases, and carbohydrate esterases. In embodiments, the enzyme is a glycoside hydrolase. For example, the glycoside hydrolase can be a beta-glucosidase. In embodiments, the enzyme is a cellulase. In embodiments, the enzyme can include a mixture of two or more CAZymes. In embodiments, the enzyme can include a mixture of one or more CAZymes with a cellulase. In embodiments, the enzyme is a lipase. For example, the enzyme can include a lipase from Candida rugosa. In general, any enzyme that has a net negative charge can be used in the biocatalysts described herein.

The assembly of the biocatalyst is believed to be due to the net negative surface charge of the enzyme, as well as hydrogen-bonding interactions between the polymer (e.g., the N of the pyridine group) and the enzyme (e.g., the H atoms in the amino acids).

The net charge of the enzyme can generally be modified by adjusting the pH of the solution. In particular, the isoelectric point (pI) of the enzyme is the pH of the solution when the net charge of the enzyme becomes zero. When the solution pH is higher than pI, the enzyme surface is mainly negatively charged. Similarly, when the pH of the solution is lower than pI, the enzyme surface is mainly positively charged. Most enzymes have a pI in the pH range of 4 to 7. Accordingly, the pH of the solution can be maintained at about 6, about 7, or higher to provide negatively charged enzymes. Thus, it is possible to form biocatalysts including a wide array of enzymes by, in part, controlling the pH of the solution.

Furthermore, without intending to be bound by theory, it is believed that weak hydrogen bonding between the pyridine group of the polymer and the enzyme (i.e., the amino acids of the enzyme) can govern the assembly between the polymer and enzyme. Unlike covalent bonding and electrostatic interactions, hydrogen bonding is a weaker interaction, which allows the enzyme to interact with the surface of the polymer core (e.g., with the pyridine group), such that the enzyme is not limited to a fixed orientation. Therefore, the enzyme can freely move near the surface of the polymer surface while maintaining and open and available active site. Moreover, the hydrophobic polymer can interact with hydrophobic portions of the enzymes via hydrophobic interactions.

The enzyme can be present in a concentration of about 0.5 mg/mL to about 20 mg/mL, for example at least about 0.5, 0.8, 1, 2, 3, 4, 5, 7, 8, 10, 12, or 15 mg/mL and/or up to about 20, 18, 16, 15, 14, 13, 10, or 8 mg/mL. In embodiments, the enzyme is present in a concentration ranging from about 1 mg/mL to about 15 mg/mL, about 2 mg/mL to about 10 mg/mL or about 4 mg/mL to about 6 mg/mL.

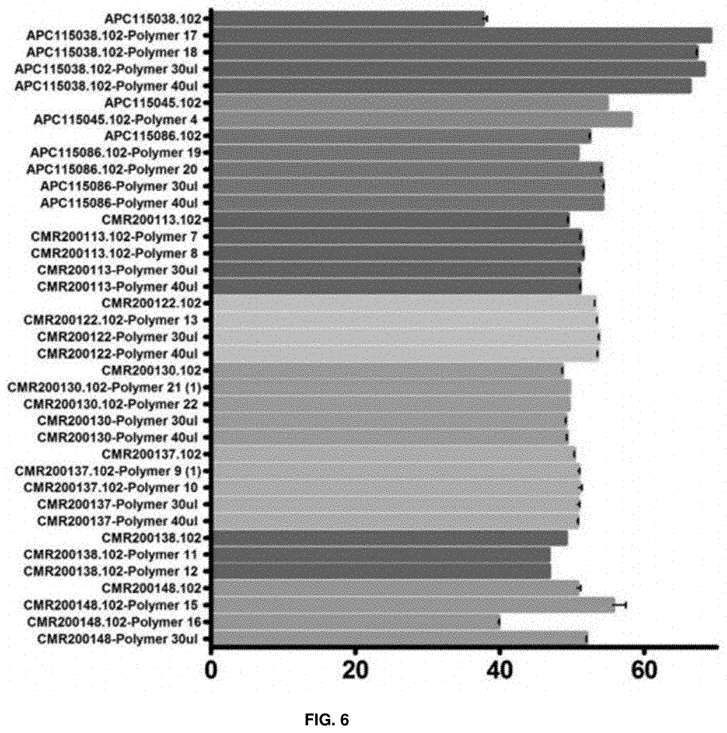

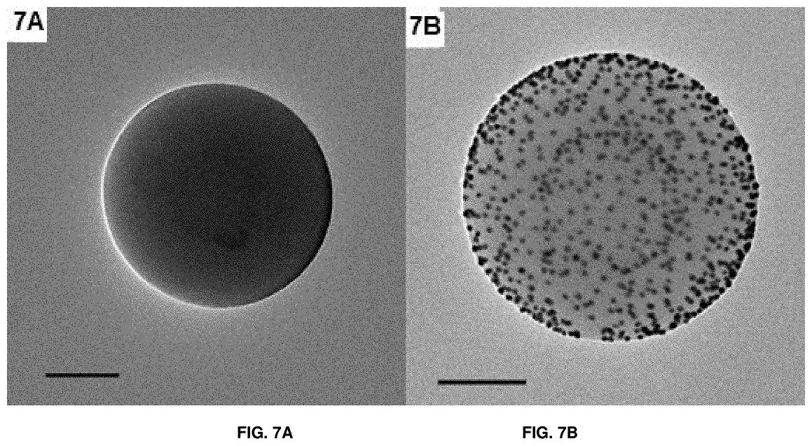

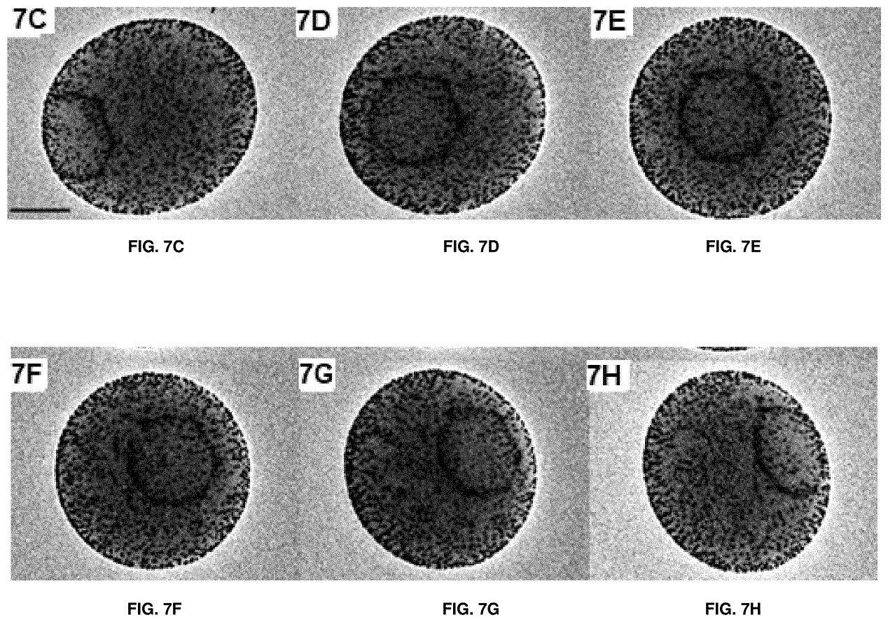

As described above, it is believed that the enzyme and polymer interact and assemble to form the shell and core of the biocatalyst, primarily through H-bonding interactions. The enzyme and polymer can interact through other interactions in addition to H-bonding. In embodiments, the enzyme covalently and/or non-covalently interacts with the polymer on the surface of the core. In embodiments, the enzyme covalently interacts with the polymer, for example, through ionic bonds. In embodiments, the enzyme non-covalently interacts with the polymer, for example through hydrogen bonds, Van der Waals interactions, hydrophobic interactions, or combinations thereof. In embodiments, the enzyme interacts with the polymer through hydrogen bonds. In embodiments, the enzyme interacts with the polymer through a combination of covalent and non-covalent bonds, for example, through hydrogen and ionic bonds. As shown in FIG. 7, the enzyme (here, demonstrated by the attachment of gold nanoparticles to provide improved visualization of the interactions) binds to the surface of the polymer core. These TEM images demonstrate that the enzyme selectively binds to and/or interacts with the polymer at the surface of the core, rather than throughout the interior of the core. Without intending to be bound by theory, it is believed that the selective interaction of the enzyme with the polymer at the surface of the core has advantageous effects on the activity and use of the biocatalyst, as the enzyme is more freely available or exposed for use.

In embodiments, the biocatalysts can have a diameter ranging from about 100 nm to about 1000 nm (1 μm), about 100 nm to about 800 nm, about 100 nm to about 600 nm, about 150 nm to about 500 nm, about 200 nm to about 400 nm, or about 250 nm to about 350 nm, for example, about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1000 nm. Other suitable diameters are contemplated herein.

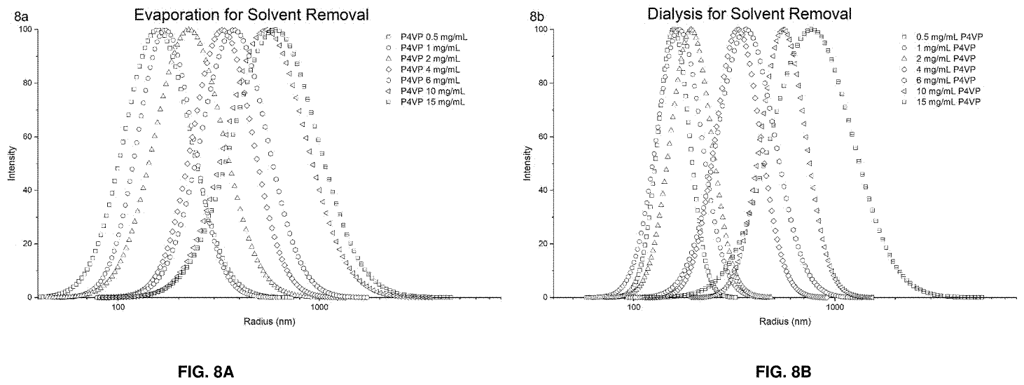

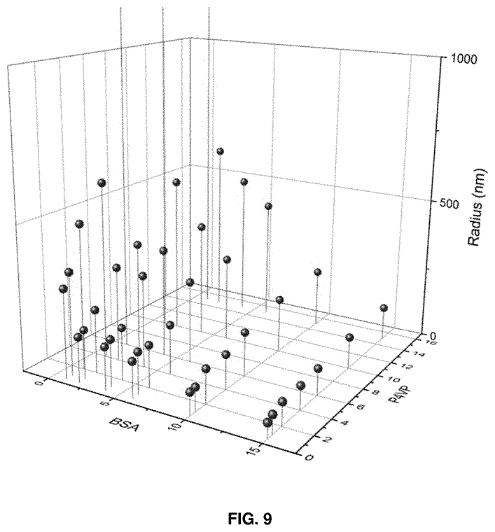

Advantageously, the diameter of the biocatalysts according to the disclosure can be controlled, in part, by the concentrations of the polymer and/or the enzyme. For example, as shown in FIG. 8A, FIG. 8B, and FIG. 9, when the enzyme concentration was kept constant (e.g., at 2.0 mg/mL) and the concentration of the polymer was varied, the radii of the resulting biocatalysts varied. Generally, as the concentration of the polymer increased, the size of the biocatalyst increased. Without intending to be bound by theory, by increasing the polymer concentration, the amount of polymer within the polymer core increased, thereby increasing the biocatalyst size.

In contrast, it was observed that when the polymer concentration remained constant and the enzyme concentration increased, the size of the biocatalyst decreased (FIG. 9). For example, as the enzyme concentration falls below 2 mg/mL, much larger biocatalysts are formed.

In embodiments, the molecular weight of the biocatalyst ranges from about 100 kDa to about 1000 kDa, about 200 kDa to about 800 kDa, about 300 to about 500 kDa, or about 350 to about 450 kDa, for example, about 100, 130, 150, 175, 200, 250, 275, 300, 350, 375, 400, 415, 450, 475, 500, 550, 600, 650, 700, 750, 775, 800, 850, 900, 950, or 1000 kDa. Other suitable molecular weights are contemplated herein. The diameter and/or molecular weight of the biocatalyst can be characterized by any method known to the person of ordinary skill in the art, for example, by dynamic light scattering (DLS) or transmission electron microscopy (TEM).

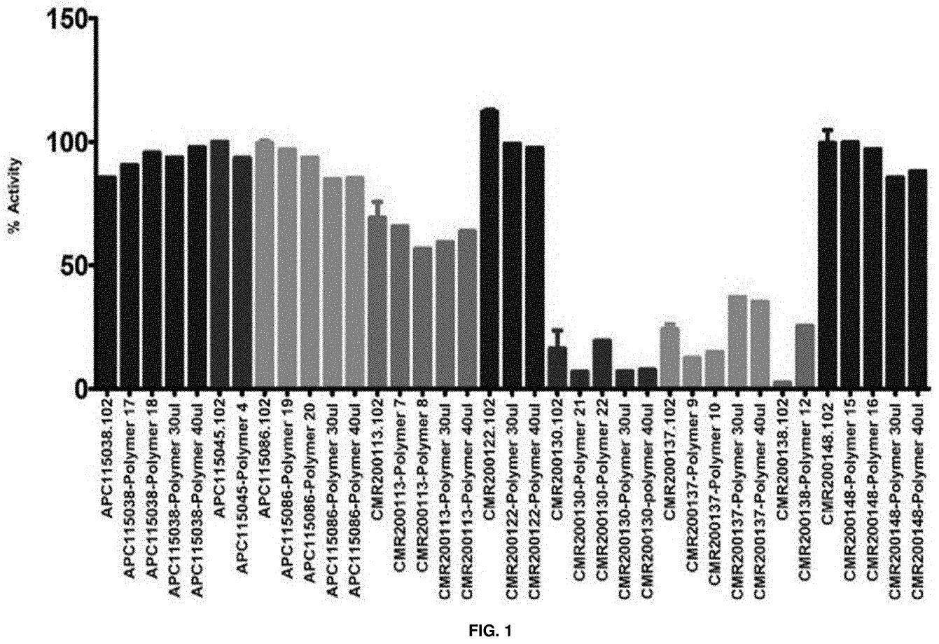

The biocatalysts of the disclosure can maintain the activity of the enzyme attached to the core. For example, the attached enzyme can have the same or substantially the same activity as that of the enzyme in a free, non-interacted state. As used herein, the term “free, non-interacted state” and “free enzyme” can be used interchangeably and refer to an enzyme as it would exist in nature. The enzyme in a free, non-interacted state (i.e., a free enzyme) can be distinguished from the enzyme of the biocatalyst, as the enzyme in the biocatalyst is bound to and interacts with the surface of the polymer core. Similarly, any reference to a “free polymer” refers to any polymer in a non-interacted or bonded state with an enzyme or other polymer. Surprisingly, after assembly, the biocatalysts (and enzymes therein) can have an activity that is at least about equal to that of the enzyme in a free, non-interacted state prior to assembly. In some cases, the biocatalysts have improved activity as compared to the enzyme in a free, non-interacted state prior to assembly. Accordingly, the biocatalysts of the disclosure have the advantageous benefit of maintaining and/or improving, enzymatic activity and stability, while also being more easily recoverable and recyclable when used in a catalytic process. As illustrated in FIG. 1, enzymes in the free, non-interacted state can have a given starting activity, and incorporation of the enzyme into the biocatalysts of the disclosure allow for maintenance of at least 80% of that starting activity and in some instances can even improve the activity of the enzyme as compared to its activity in the free, non-interacted state. In embodiments, the biocatalysts have an activity that is at least about 80% of the activity of the enzyme in a free, non-interacted state. For example, the biocatalyst activity can have an activity that is at least about 80%, 85%, 90%, 95%, 100%, 105%, 110%, or 115% of the activity of the enzyme in a free, non-interacted state.

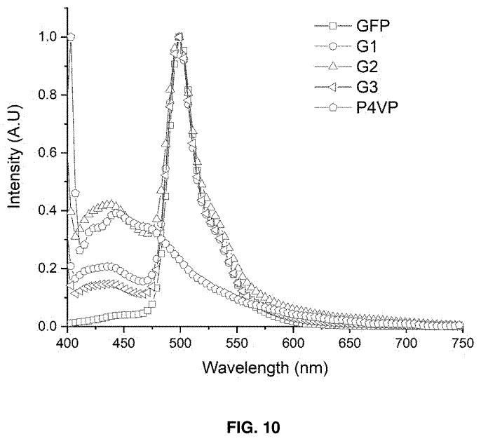

The biocatalysts of the disclosure can maintain the stability of the enzyme upon attachment of to the core. For example, the stability of the enzyme on biocatalysts of the disclosure can be the same or substantially the same as the stability of the enzyme in a free, non-interacted state. For example, as shown in FIG. 10, when a green fluorescent protein (GFP) was used in place of an enzyme (e.g., to provide a quantifiable representation of stability upon formation), no change in the emission spectra of the protein at various biocatalyst sizes (i.e., 500 nm, 750 nm, and 900 nm) was observed relative to the “free” GFP. When GFP undergoes denaturing it loses its fluorescence. Thus, the maintained fluorescence shown in FIG. 10 demonstrates that formation of the biocatalysts according to the disclosure does not result in the denaturation of the enzymes, and rather results in the maintenance or improvement in enzymatic stability.

Surprisingly and advantageously, the biocatalysts of the disclosure have a melting point that is at least equal to the melting point of the enzyme in a free, non-interacted state. Without intending to be bound by theory, it is believed that the ability of the biocatalyst to maintain the melting point of the enzyme in a free, non-interacted state indicates that the enzyme structures at the polymer surface are stable. In some cases, the melting point of the biocatalyst is much greater than that of the corresponding enzyme in a free, non-interacted state, demonstrating enhanced stability of the enzyme at the polymer surface, as compared to its free form.

In some embodiments, the enzymatic activity of the biocatalyst after storage for 200 days at room temperature is at least about equal to the enzymatic activity of the enzyme in a free, non-interacted state.

### Methods of Preparing Biocatalyst

In accordance with embodiments, a method of forming a biocatalyst can include admixing an aqueous phase comprising an enzyme with a dispersed phase comprising a polymer under conditions sufficient to disperse droplets of the dispersed phase in the aqueous phase, wherein the enzyme interacts with the polymer and arranges at the interface between the droplets of the dispersed phase and the aqueous phase to form the shell. The conditions in which the enzyme interacts with the polymer and arranges to form the shell are such that the enzyme remains active (i.e., maintains at least 80% activity) after forming the shell. In embodiments, the polymer has a pyridine functional group. In embodiments, the enzyme is one or more carbohydrate-active enzyme, cellulase, and any mixture or combination thereof.

In nature, biocatalysts can be used under a variety of conditions, such as in anaerobic or aerobic environments, as well as variable salt and pH environments. Generally, enzymes have amino acids on their surface to accommodate these environmental factors and can be more acidic, basic, and/or hydrophobic depending on the environment. Therefore, as would be appreciated by the skilled artisan, the particular methods (e.g., buffer pH, temperature, salt, etc.) of preparing the biocatalysts can depend on the particular enzyme in the biocatalyst. For example, in some cases the isoelectric and/or ionic strength of the admixture (e.g., the buffer) must be controlled depending on the particular enzyme.

Without intending to be bound by theory, it is believed that the water-soluble enzyme aids in stabilizing a hydrophobic polymer in an aqueous environment to reduce the surface energy of the polymer, thereby forming thermodynamically stable biocatalysts.

In embodiments, the dispersed phase includes the polymer dissolved in a solvent. Various solvents can be used and selected as known in the art given the particular polymer. For example, suitable solvents for dissolving the polymer include, but are not limited to, water, ethanol, methanol, dimethylformamide (DMF), dimethylsulfoxide (DMSO), tetrahydrofuran (THF), or any combination thereof. In some embodiments, the polymer is dissolved in a solvent selected from the group consisting of water, ethanol, methanol, dimethylformamide (DMF), dimethylsulfoxide (DMSO), tetrahydrofuran (THF), and any combination thereof. In some embodiments, the polymer is dissolved in methanol.

In embodiments, upon admixing the aqueous phase with the dispersed phase, the admixture can have less than about 50 vol. % solvent, based on the total volume of the admixture. For example, in embodiments, the admixture can have less than about 50, 45, 40, 35, 30, 25, 20, 15, 10, or 5 vol. % solvent based on the total volume of the admixture. In some embodiments, the admixture can have less than about 30 vol. % solvent, based on the total volume of the admixture.

In embodiments, the method can be carried out at various temperatures. Advantageously, in embodiments, the method can be carried out at room temperature. Other suitable temperatures include about 20° C. to about 80° C., about 25° C. to about 75° C., about 35° C. to about 65° C., or about 40° C. to about 50° C. For example, the temperature can be about 20, 25, 30, 35, 40, 45, 50, 55, 60, 65, 70, 75, or 80° C.

In embodiments, the admixing occurs under a pH ranging from about 6 to about 12, about 7 to about 10, or about 8 to about 9, for example about 6, 6.5, 7, 7.5, 8, 8.5, 9, 9.5, 10, 10.5, 11, 11.5, or 12.

After formation of the biocatalysts, the solvent can be removed, for example, through evaporation or dialysis. Advantageously, dialysis allows for removal of unreacted—or free—enzyme and polymer from the admixture, as the dialysis conditions (e.g. the pore size of the dialysis tube) can be selected such that any enzyme in a free, non-interacted state and/or free polymer (i.e., not in the core) can be removed from the admixture, while the biocatalyst, having a higher molecular weight and diameter relative to each of the enzyme in a free, non-interacted state and free polymer remains in the dialysis tube. The results shown in FIGS. 8A and 8B show the consistency in biocatalyst size between the two methods. As mentioned, dialysis allows for the removal of any unreacted or free enzyme along with the solvent. However, there is a higher cost associated with the dialysis equipment along with effort involved in changing the dialysis solution at the required intervals. Evaporation is the simpler solvent removal method, but can result in unreacted or free enzyme remaining in the solution.

Advantageously, in embodiments, the stability of the biocatalysts does not demonstrate an electrostatic effect. That is, the stability of the biocatalysts is not diminished by the addition of salts or other electrolytes. Accordingly, in embodiments, the admixture can further include a salt. In embodiments, the concentration of salt can be about 25 μM to about 250 μM, about 50 μM to about 200 μM, or about 100 μM to about 150 μM, for example about 25, 50, 100, 150, 200, or 250 μM. In some embodiments, the salt has a concentration of about 250 μM. Various salts can be used, including, but not limited to sodium chloride, potassium chloride, lithium chloride, magnesium chloride, calcium chloride, and the like. In some embodiments, the salt is sodium chloride.

The biocatalysts and methods in accordance with the disclosure can be better understood in light of the following examples, which are merely intended to illustrate the compositions and methods, and are not meant to limit the scope thereof in any way.

### EXAMPLES

### Example 1: Preparation and Evaluation of the Biocatalyst

A solution of P4VP in methanol was slowly added into a solution of enzyme in pure water under stirring. The identity and reference names for each enzyme are displayed in Table 1, below.

TABLE 1
Evaluated Enzymes

| Enzyme | |
|---|---|
| Reference | Enzyme Type (GenBank Accession No.) |
| APC115038 | Glycosyl Hydrolase Family 3 N-Terminal Domain Protein (EEF90995) |
| APC115045 | Glycosyl Hydrolase Family 3 N-Terminal Domain Protein (EDV07201) |
| APC115086 | Glycosyl Hydrolase Family 3 N-Terminal Domain Protein (EDV06546) |
| CMR200113 | Bacteroides intestinalis DSM 17393; cellulase |
| CMR200122 | Bacteroides intestinalis periplasmic beta-glucosidase |
| CMR200130 | Bacteroides plebeius DSM 17135; beta-galactosidase |
| CMR200137 | Bacteroides plebeius; beta-galactosidase |
| CMR200138 | Bacteroides plebeius; beta-galactosidase |
| CMR200148 | Bacteroides plebeius; periplasmic beta-glucosidase precursor |

The resulting biocatalysts were characterized via dynamic light scattering (DLS) using the DynaPro Plate Reader with Dynamics 6.10.1.2 program. Table 2 shows the average molecular weights of the biocatalysts.

TABLE 2
Average Molecular Weights of Evaluated Enzymes

| Enzyme Reference | Average Mw (kDa) |
|---|---|
| APC115038 | 414 ± 211 |
| APC115045 | 160 ± 16 |
| APC115086 | 246 ± 56 |
| CMR200113 | 159 ± 43 |
| CMR200122 | 137 ± 24 |
| CMR200130 | 287 ± 123 |
| CMR200137 | 765 ± 371 |
| CMR200148 | 389 ± 196 |

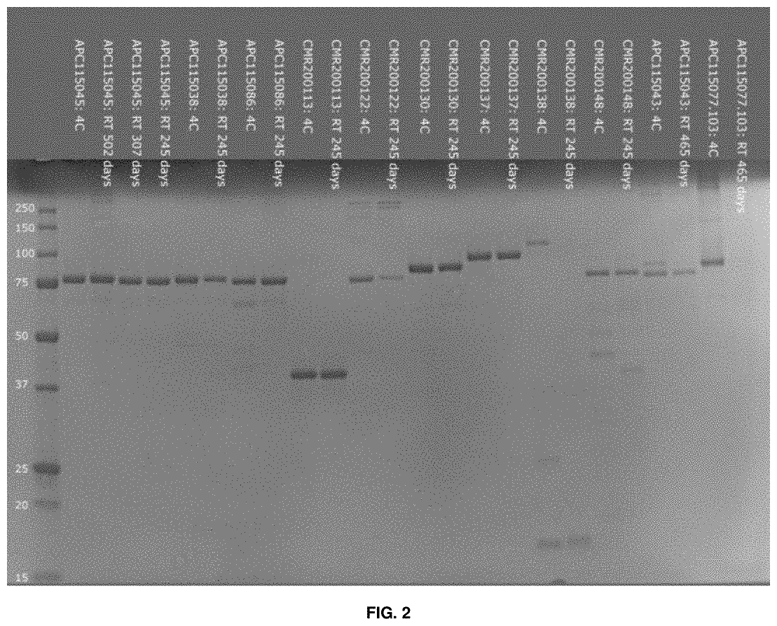

The activity of the enzyme-polymer biocatalyst was measured and compared to the activity of the enzyme in a free, non-interacted state. As shown in FIG. 1, compared to the enzyme in a free, non-interacted state, the biocatalyst maintains substantially the same activity. For one enzyme in particular, CMR200138, the enzymatic activity is significantly improved when used in the biocatalyst of the disclosure. This enzyme, when present as an enzyme in a free, non-interacted state, has little stability at room temperature, as shown in FIG. 2, thereby indicating that the biocatalyst helped to stabilize the enzyme from degradation.

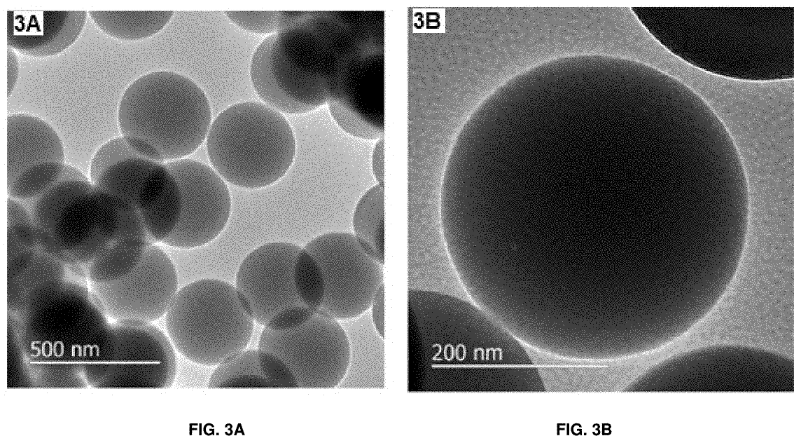

The resulting biocatalysts were also imaged using transmission electron microscopy (TEM). Images of the resulting biocatalysts, having an average diameter of about 420 nm with a narrow size distribution are shown in FIG. 3.

### Example 2: Effect of Salt Concentration on Biocatalyst Activity

The effect of salt on enzyme activity was tested by using 6 different concentrations of NaCl in a pH 6.8 HEPES buffer. The salt ranged from 25 μM to 250 μM NaCl. Fluorescein Di-3-D-Glucopyranoside (FDGlu) was used as the substrate. APC115045.102 (30 nM) was set up in triplicate, in each of the 6 salt concentration buffers with 50 μM FDGlu. All reactions were set up in a 96 well Costar black with clear bottom plate. The plate was read on a SpectraMax fluorescent plate reader kinetically for 3 hours at 5 minute intervals, with the wavelengths as follows: excitation: 485 nm, cutoff: 495 nm, emission: 515 nm. The concentrations of NaCl in the buffers were: 250 μM, 200 μM, 150 μM, 100 μM, 50 μM, 25 μM.

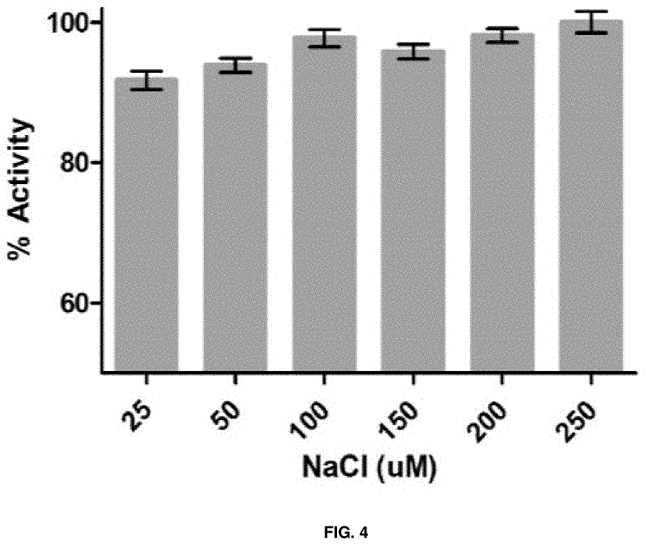

As shown in FIG. 4, the enzyme was most active at a salt concentration of 250 μM. Example 2 demonstrates that the biocatalysts maintain activity in the presence of increased electrolytic interactions, such as from NaCl.

### Example 3: Effect of Heating on Biocatalyst Activity

The effect of the temperature on biocatalyst activity was explored and compared with the activity of the enzyme in a free, non-interacted state.

Each enzyme in a free, non-interacted state or enzyme-polymer biocatalyst sample was set up in triplicate wells on a Phenix plate at 60 nM in enzyme buffer (10 mM HEPES pH 6.8, 250 mM NaCl). The plates were then heated to 25° C., 40° C., 60° C., or 80° C. in a thermocycler for 5 minutes. The plates were cooled on ice then held at 4° C. until they could be set up with FDGlu.

All reactions were set up in a 96 well Costar black with clear bottom plate. Each reaction contained 30 nM enzyme in a free, non-interacted state or enzyme-polymer biocatalyst and 50 μM FDGlu. The plate was read on a SpectraMax fluorescent plate reader kinetically for 3 hours at 5 minute intervals, with the wavelengths as follows: excitation: 485 nm, cutoff: 495 nm, emission: 515 nm.

The enzyme-polymer biocatalysts maintained 100% activity at 40° C. The activity of the biocatalyst dropped to 0.003% at 60° C. Accordingly, Example 3 demonstrates that the Biocatalysts are Thermally Stable Up to Temperatures of at Least 40° C.

### Example 4: Effect of Solvent and Solvent Concentration on Enzyme Activity

The effect of the identity and concentration of the solvent on the enzyme activity of the enzyme-polymer biocatalyst of Example 1 was explored. Five solvents were tested: EtOH, MeOH, DMF, DMSO, and THF, each at various concentrations. The enzyme concentration was 30 nM and the FDGlu concentration was 50 μM. All reactions were carried out in buffer (10 mM HEPES pH 6.8, 250 mM NaCl) and the specified solvent.

All reactions were set up in a 96 well Costar black with clear bottom plate. The plate was read on a SpectraMax fluorescent plate reader kinetically for 3 hours at 5 minute intervals with the wavelengths as follows: excitation: 485 nm, cutoff: 495 nm, emission: 515 nm.

The activity of the enzyme in a free, non-interacted states and enzyme-polymer biocatalysts were tested at the following solvent concentrations:

Ethanol: 5% to 90% by volume;

Methanol: 5% to 60% by volume;

DMSO: 5% to 50% by volume; and,

THF and DMF: 5% to 40% by volume.

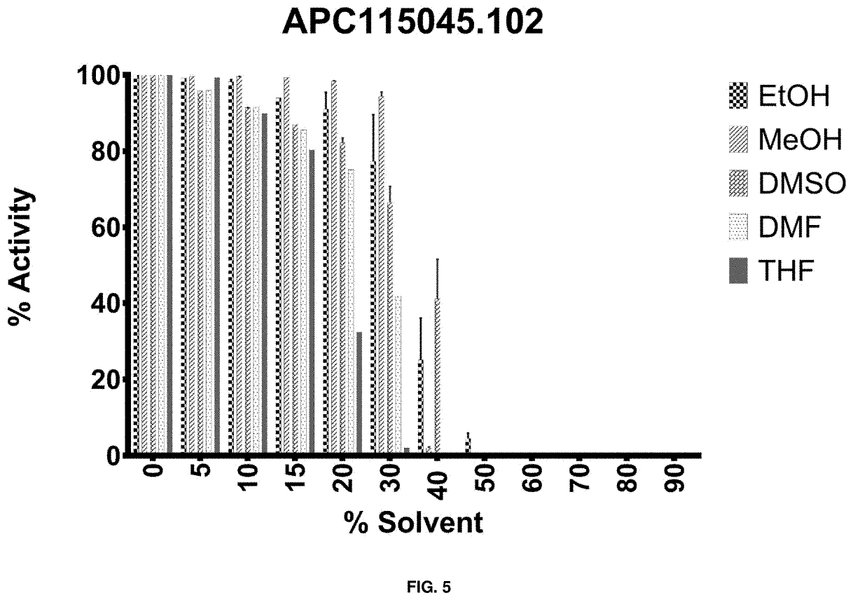

The activity of the enzyme of the biocatalyst in each of the tested solvents is shown in FIG. 5. Surprisingly and advantageously, the enzyme of the biocatalyst maintained its activity (˜95% active) at a methanol concentration of 30% by volume.

Example 4 demonstrates that the biocatalysts of the disclosure can withstand greater organic solvent concentrations than the enzyme in a free, non-interacted state.

### Example 5: Effect of pH on Enzyme Activity

The effect of buffer pH on enzyme activity was explored.

Four buffers were used to test the activity of enzymes for biocatalysts made in accordance with Example 1 and including the enzyme APC115045. The buffers were all prepared at 20 mM concentrations and the pH was adjusted. The buffers tested were: sodium acetate pH 4; MES pH 6, HEPES pH 8, and CHES pH 10. Each reaction contained 30 nM enzyme in a free, non-interacted state or enzyme-polymer biocatalyst and 50 μM FDGlu. Each sample was set up in triplicate in each of the four buffers. All reactions were set up in a 96 well Costar black with clear bottom plate. The plate was read on a SpectraMax fluorescent plate reader kinetically for 2 hours at 5 minute intervals, with the wavelengths as follows: excitation: 485 nm, cutoff: 495 nm, emission: 515 nm. No salt was added to any of the buffers.

The enzyme on the biocatalyst had 100% activity in the HEPES pH 8 buffer.

Accordingly, Example 5 demonstrates that the biocatalysts are stable at neutral to alkaline pH.

### Example 7: Melting Point Stability of Biocatalyst

The melting points of the enzyme in a free, non-interacted states and enzyme-polymer biocatalysts were explored through the fluorescent thermal shift. Biocatalysts were set up in triplicate at 0.5 μM enzyme concentration with 1× Sypro Orange in 10 mM HEPES pH 6.8, 250 mM NaCl buffer. The samples were tested on the CFX plate reader from 25° C. to 95° C., increasing by 0.5° C. every 30 seconds.

In general, at temperatures below the melting point of the enzyme in a free, non-interacted state, the enzyme in a free, non-interacted states are stable. However, as the temperatures increase above the melting point, enzyme in a free, non-interacted states lose activity. As shown in FIG. 6, the melting points of the biocatalysts were generally equivalent to the melting points of the enzyme in a free, non-interacted states, indicating that the enzyme structure at the polymer surface in the biocatalyst maintained the stability of the enzyme in a free, non-interacted state. However, for APC115038, the melting point of the biocatalyst was much higher than the enzyme in a free, non-interacted state, indicating improved stability of the biocatalyst over the enzyme in a free, non-interacted state.

### Example 8: Effect of Solvent Removal Method

Removal of the solvent was performed via evaporation and dialysis.

Evaporation was performed in open air at 25° C. while stirring. The results are shown in FIG. 8A.

Dialysis was performed using FLOAT-A-LYZER® G2 dialysis devices purchased from Spectrum Labs. All dialysis devices were pre-treated via a 10% ethanol bath for ten minutes before thoroughly rinsing in DI H₂O, per manufacturer instructions. Each sample was then transferred to its own dialysis tube and underwent dialysis against a 1.0 L 10 mM HEPES (pH 8) and 250 mM NaCl solution. The dialysis solution was replaced by a fresh solution after 4, 6, 10, and 12 hours. Once dialysis was complete, the solution containing the biocatalysts was then retrieved from the dialysis tube via pipette.

It was found that the method used for removing the solvent can slightly impact the size of the biocatalysts. Furthermore, dialysis allowed for the removal of unreacted enzyme in a free, non-interacted state, thereby ensuring that all of the enzyme in the biocatalyst was anchored to the surface of the polymer core.

### Example 9: Effect of Varying Polymer and Enzyme Concentrations

The impact that the concentrations of polymer and enzyme had on the size of the biocatalysts was evaluated.

Biocatalysts were prepared with the enzyme and polymer concentrations shown in Table 3, where bovine serum albumin (BSA) was used as a model protein. Each reaction was initiated by adding P4VP (0.36 mL) in MeOH dropwise in three 0.12 mL increments to BSA (1.0 mL) in a 3.7 mL glass vial. The vial was then sealed for thirty minutes before being transferred to a 1000 kD dialysis tube and placed in the dialysis solution. Dialysis was performed using Float-A-Lyzer®G2 dialysis devices purchased from Spectrum Labs. All dialysis devices were pre-treated via a 10% ethanol bath for ten minutes before thoroughly rinsing in DI H2O, per manufacturer instructions. Each sample is then transferred to its own dialysis tube and underwent dialysis against a 1.0 L 10 mM HEPES and 250 mM NaCl solution (pH 6.95). The dialysis solution was replaced by a fresh solution after 4, 6, 10, and 12 hours. Once dialysis was complete, the solution containing the biocatalysts was then retrieved from the dialysis tube via pipette. Each reaction shown in Table 3 follows the above protocol, with the only variance being the changes in P4VP and BSA concentrations.

TABLE 3
Polymer and Enzyme Concentrations and
Resulting Biocatalyst Size

| Biocatalyst | | P4VP Concentration (mg/mL) | | | | | | |
|---|---|---|---|---|---|---|---|---|
| Radius (nm) | | 0.5 | 1.0 | 2.0 | 4.0 | 6.0 | 10.0 | 15.0 |
| BSA | 1.0 | 310 | 356 | 500 | 609 | 1334 | 4001 | 4208 |
| Concentration | 2.0 | 157 | 171 | 221 | 329 | 377 | 543 | 603 |
| (mg/mL) | 4.0 | 149 | 163 | 181 | 321 | 374 | 396 | 497 |
| | 6.0 | 126 | 146 | 156 | 174 | 284 | 295 | 419 |
| | 10.0 | 78 | 82 | 118 | 124 | 153 | 189 | 202 |
| | 15.0 | 52 | 65 | 80 | 84 | 92 | 110 | 118 |

It was observed that as the concentration of the polymer increased, the size of the biocatalyst similarly increased. In contrast, it was observed that as the concentration of the enzyme increased, the size of the biocatalyst decreased. Thus, Example 9 demonstrates that the size of the biocatalyst can be controlled, in part, by the concentrations of the polymer and/or enzyme.

### Example 10: Enzyme Stability

The stability of the enzyme in the biocatalysts was evaluated by using a green fluorescent protein (GFP) as a model for the enzyme. GFP is known to lose its fluorescence upon denaturation, and therefore was selected as a model protein to easily discern via the resulting fluorescence spectra if the enzyme denatured upon formation of the biocatalyst.

Synthesis of three variations of GFP biocatalysts were as follows. Each biocatalyst had a different size: G1 had a diameter of 500 nm, G2 had a diameter of 750 nm, and G3 had a diameter of 900 nm.

For sample G1, a solution containing P4VP (Mw 60 kDa) in DMF (2.0 mg/mL, 0.05 mL) was added dropwise, in 15, 15, and 204 increments, to a 3.7 mL glass vial containing the GFP (0.8 mg/mL, 0.5 mL) in a 20 mM tris(hydroxymethyl)aminomethane (Tris) pH 7.0, 80 mM NaCl, and 2 mM EDTA buffer solution. The solution was constantly stirred during addition of P4VP. The vial was then sealed for thirty minutes before undergoing dialysis. Dialysis was done in a 1.0 L 10 mM HEPES and 250 mM NaCl solution. The dialysis solution was replaced by a fresh solution after 4, 6, 10, and 12 hours. Once dialysis was complete, the solution containing the biocatalysts was then retrieved from the dialysis tube via pipette. All samples in this research were synthesized using the above procedure with only variations to the amount of P4VP that was added. P4VP volumes of 0.12 and 0.20 mL were used for G2 and G3, respectively.

Fluorescence spectra were obtained for each of G1, G2, and G3, as well as free GFP and free P4VP. The spectra are shown in FIG. 10. As shown in this figure, the formation of the biocatalyst did not result in the denaturation of the GFP, as the fluorescence of the GFP in each of the biocatalysts was at the same intensity and wavelength of the free GFP. Because the solvent was removed via dialysis, thereby allowing for the removal of free GFP, it can be concluded that the fluorescence of each of G1, G2, and G3 was due to GFP bound to the surface of the polymer core.

Therefore, Example 10 shows that the enzyme remains as stable when in the biocatalyst as it does as an enzyme in a free, non-interacted state.

### Example 11: Formation of Lipase Biocatalysts

The biocatalysts were prepared using a lipase (i.e., as the enzyme) from Candida rugosa.

For the lipase (Candida rugosa) biocatalysts, a 2.0 mg/mL solution was prepared by dissolving the lyophilized enzyme in a 10 mM acetate buffer (pH 4.5). Then, a solution containing P4VP (Mw 60 kDa) in MeOH (2.0 mg/mL, 0.12 mL) was added dropwise, in 404 increments, to a 3.7 mL glass vial containing the lipase (0.8 mg/mL, 0.5 mL). The solution was constantly stirred during addition of P4VP. The vial was then sealed for thirty minutes before undergoing dialysis. The lipase reactions were dialyzed against a 1.0 L 10 mM acetate buffer (pH 4.5) in a 1000 kD dialysis tube. The dialysis solution was replaced by a fresh solution after 4, 6, 10, and 12 hours. Once dialysis was complete, the solution containing the biocatalysts was then retrieved from the dialysis tube via pipette. All samples in this Example were synthesized using the above procedure with only variations to the polymer concentration.

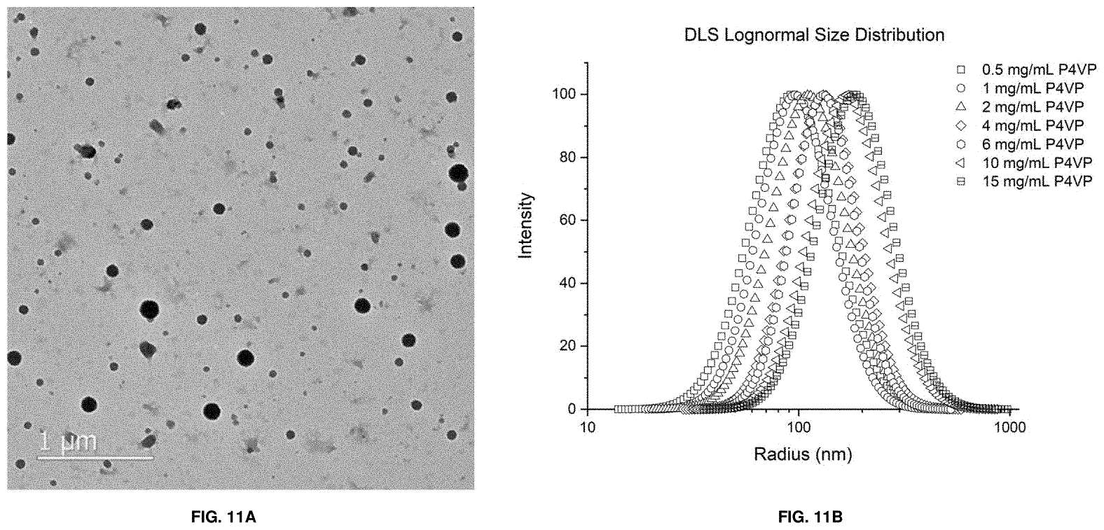

As shown in FIG. 11A, the lipase enzymes were successfully incorporated into the biocatalysts. Moreover, as shown in FIG. 11B, the size of the biocatalyst was easily controlled by varying the amounts of polymer.

Thus, Example 11 demonstrates that the formation of the biocatalysts is not limited to any particular enzyme and can be used with many different classes and types of enzymes.

1. A method of forming a biocatalyst having a core surrounded by a shell, the method comprising:
admixing an aqueous phase comprising an enzyme with a dispersed phase comprising a polymer having a pyridine functional group under conditions sufficient to disperse droplets of the dispersed phase in the aqueous phase,
wherein the enzyme interacts with the polymer and arranges at the interface between the droplets of the dispersed phase and the aqueous phase to form the shell, and wherein after forming the shell, the enzyme has an activity that is at least 80% of an activity of the enzyme in a free, non-interacted state.

2. (canceled)

3. The method of claim 1, wherein the polymer having a pyridine functional group is selected from the group consisting of poly(styrene-b-4-vinyl pyridine), poly(styrene-b-2-vinyl pyridine), poly(2-vinyl pyridine-b-ε-caprolactone), poly(ethylene oxide-b-4-vinyl pyridine), poly(styrene-b-4-vinyl pyridine-b-styrene), poly(4-vinyl pyridine-b-styrene-b-4-vinyl pyridine), poly(styrene-b-4-vinyl pyridine-b-ethylene oxide), and any combination thereof.

4. The method of claim 1, wherein the enzyme has a net negative surface charge.

5. (canceled)

6. The method of claim 1, wherein the enzyme interacts with the polymer through hydrogen bonds.

7. (canceled)

8. The method of claim 1, wherein the dispersed phase comprises the polymer dissolved in a solvent, wherein the solvent is one or more of water, ethanol, methanol, dimethylformamide (DMF), dimethylsulfoxide (DMSO), tetrahydrofuran (THF), or any combination thereof.

9. (canceled)

10. (canceled)

11. The method of claim 1, wherein the admixing occurs at a temperature ranging from about 20° C. to about 80° C.

12. The method of claim 1, wherein the admixture further comprises a salt, wherein the salt is present in a concentration ranging from about 25 μM to about 250 μM.

13. (canceled)

14. (canceled)

15. The method of claim 1, wherein the enzyme is selected from the group consisting of carbohydrate-active enzymes, cellulase, lipase, and any mixture or combination thereof

16. (canceled)

17. (canceled)

18. (canceled)

19. (canceled)

20. (canceled)

21. (canceled)

22. The method of claim 1, wherein the biocatalyst has a diameter ranging from about 100 nm to about 1000 nm.

23. (canceled)

24. (canceled)

25. (canceled)

26. (canceled)

27. (canceled)

28. A biocatalyst comprising:
a core comprising a polymer having a pyridine functional group, and
a shell comprising an enzyme,
wherein the enzyme covalently and/or non-covalently interacts with the polymer on the surface of the core, and the biocatalyst has (a) a melting point that is at least equal to the melting point of the enzyme in a free, non-interacted state, and (b) an enzymatic activity after 200 days at room temperature that is at least about equal to the enzymatic activity of the enzyme in a free, non-interacted state.

29. (canceled)

30. The biocatalyst of claim 28, wherein the polymer having a pyridine functional group is selected from the group consisting of poly(styrene-b-4-vinyl pyridine), poly(styrene-b-2-vinyl pyridine), poly(2-vinyl pyridine-b-ε-caprolactone), poly(ethylene oxide-b-4-vinyl pyridine), poly(styrene-b-4-vinyl pyridine-b-styrene), poly(4-vinyl pyridine-b-styrene-b-4-vinyl pyridine), poly(styrene-b-4-vinyl pyridine-b-ethylene oxide), and any combination thereof.

31. The biocatalyst of claim 28, wherein the enzyme has a net negative surface charge.

32. The biocatalyst of claim 28, wherein the enzyme interacts with the polymer through hydrogen bonds.

33. (canceled)

34. The biocatalyst of claim 28, wherein the enzyme is selected from the group consisting of carbohydrate-active enzymes, cellulase, lipase, and any mixture or combination thereof

35. (canceled)

36. (canceled)

37. (canceled)

38. (canceled)

39. (canceled)

40. (canceled)

41. The biocatalyst of claim 28, wherein the polymer has a molecular weight ranging from about 20 kDa to about 250 kDa.

42. The biocatalyst of claim 28, wherein the polymer is present in a concentration of about 0.1 mg/mL to about 20 mg/mL.

43. (canceled)

44. The biocatalyst of claim 28, wherein the biocatalyst has a diameter ranging from about 100 nm to about 1000 nm.

45. (canceled)

46. The biocatalyst of claim 28, wherein the biocatalyst has a molecular weight of about 100 kDa to about 1000 kDa.

47. The biocatalyst of claim 28, wherein the biocatalyst has a melting point that is equal to or greater than the melting point of the enzyme in a free, non-interacted state.

48. The biocatalyst of claim 28, wherein the biocatalyst has an enzymatic activity that is at least about equal to the enzymatic activity of the enzyme in a free, non-interacted state.
